(19) United States
(12) Patent Application Publication
(10) Pub. No.: US 2025/0325745 A1
(43) Pub. Date: Oct. 23, 2025

(54) ROTATING INTRAVENOUS NEEDLES AND CATHETERS

(71) Applicant: David Ethington, Blackfoot, ID (US)

(72) Inventor: David Ethington, Blackfoot, ID (US)

(21) Appl. No.: 18/638,434

(22) Filed: Apr. 17, 2024

(51) Int. Cl. A61M 5/158; A61M 25/06

(52) U.S. Cl. CPC A61M 5/158; A61M 25/0606; A61M 2005/1585; A61M 2005/1588; A61M 2205/103; A61M 2205/3327; A61M 2205/586

(57) ABSTRACT

Described herein are examples of devices which include a rotating intravenous needle and catheter including a needle housing, an intravenous needle, a catheter, a rotation actuator, and a tension system, wherein the device is configured to rotate the intravenous needle 180° when placed inside a patient in order to reduce the potential for puncturing vein of the patient.

### BACKGROUND

Intravenous needles are ubiquitous component of medical care for millions of people. Particularly in hospital settings intravenous needles with and without catheters are routinely employed on patients for multiple types of treatments. Intravenous needles and catheter equipment must be sanitary, reliable and cost effective to safely and efficiently treat patients. Intravenous (IV) lines, arterial lines, and peripherally inserted central catheter (PICC) lines are different types of lines places into a vein or artery for medical care.

### BRIEF DESCRIPTION OF THE DRAWINGS

The present description will be understood more fully when viewed in conjunction with the accompanying drawings of various examples of rotating intravenous needles and catheters. The description is not meant to limit the rotating intravenous needles and catheters to the specific examples. Rather, the specific examples depicted and described are provided for explanation and understanding of rotating intravenous needles and catheters. Throughout the description the drawings may be referred to as drawings, figures, and/or FIGs.

FIG. 1 illustrates a top view of a rotating intravenous needle and catheter, according to an embodiment.

FIG. 2 illustrates a side view of a rotating intravenous needle and catheter, according to an embodiment.

FIG. 3 illustrates an alternative top view of a rotating intravenous needle and catheter, according to an embodiment.

FIG. 4 illustrates an alternative side view of a rotating intravenous needle and catheter, according to an embodiment.

FIG. 5 illustrates a block chart of a method of administering a rotating intravenous needle and catheter to a patient, according to an embodiment.

### DETAILED DESCRIPTION

Rotating intravenous needles and catheters, arterial line needles, and peripherally inserted central catheter (PICC) line needles as disclosed herein will become better understood through a review of the following detailed description in conjunction with the figures. The detailed description and figures provide merely examples of the various embodiments of rotating intravenous needles and catheters. Many variations are contemplated for different applications and design considerations; however, for the sake of brevity and clarity, all the contemplated variations may not be individually described in the following detailed description. Those skilled in the art will understand how the disclosed examples may be varied, modified, and altered and not depart in substance from the scope of the examples described herein.

Conventional intravenous needles are stationary, i.e., they do not have the ability to rotate the needle beyond physical turning of the needle housing by the administrator such as doctors, nurse, or other medical personnel. Without the ability to easily rotate the needle after insertion into the patient the needle can potentially puncture the vein in which it has been inserted. This is due to the bevel nature of the tip of standard intravenous needles. A common practice is to rotate the needle after insertion thereby rotating the bevel and reducing the potential for puncturing the patients vein.

Additionally, dehydrated patients, patients with a history of drug use, obesity, as well as young children and geriatric patients can have veins that are difficult to properly place the intravenous needle and catheter. These patients can have veins that are too small in diameter, thinned vein walls, easily collapsing veins or hard to find veins. The ability to rotate the needle after insertion can improve the patient's experience by reducing the possible pain associated with puncturing the vein as well as the additional discomfort of having to administer the intravenous needle and catheter.

Currently the way to rotate the needle is to physically turn the needle housing. However, in standard practice the administrator will often be manipulating the needle with one hand while using the other hand to attend to the patient, hold other equipment or materials, and/or remove the catheter from the needle housing. This can lead to extreme difficulty in properly rotating the needle and or unintentional puncturing of the patient's vein while manipulating the needle housing.

Existing intravenous needles and catheter equipment do not provide a device or system for safely and efficiently rotating an intravenous needle after insertion into a patient. There is a need for a cost effective, safe, and efficient device or system to rotate a needle after insertion into a patient.

Implementations of a rotating intravenous needle and catheter may address some or all of the problems described above, and such implementations may lead to improved patient outcome, faster and more reliable treatment and cost and time savings to patients and medical facilities.

Embodiments of rotating intravenous needles and catheters may include tension systems to rotate the needle via compression of levers on the side of the needle housing. Alternatively, the tension system can be internal in the needle housing and operated via a button or tab located on the exterior of the needle housing. The tension system can include gears, springs, and other resistance materials.

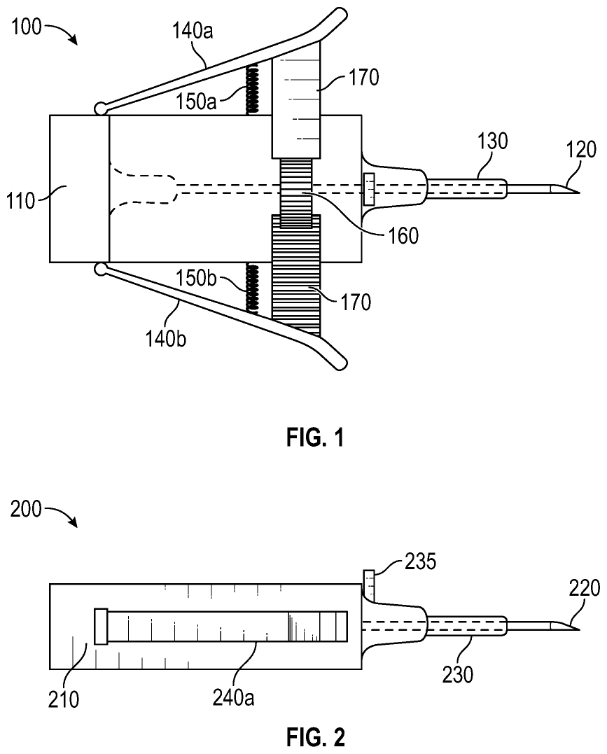

FIG. 1 illustrates a top view of a rotating intravenous needle and catheter 100, according to an embodiment. The rotating intravenous needle and catheter 100 can allow an administrator to safely and efficiently insert a needle into a patient with the ability to rotate the bevel of the needle to provide additional safety for the patient.

The rotating intravenous needle and catheter 100 can include a needle housing 110, a needle 120, a catheter 130, levers 140, springs 150, gears 160, and gear tracks 170. The needle housing 110 is generally cylindrical and configured to be held in the hand of the administrator during insertion of the needle and catheter into the patient. The needle housing 110 can include ridges or grips to ensure a firm grip by the administrator. The needle housing 110 can also include a bevel indicator (not shown) to inform, show or indicate to the administrator the angle of rotation of the needle 120.

Embodiments of the needle housing 110 and catheter 130 can be prepared from medical grade plastics and polymers. Embodiments of the needle 120 can be prepared from stainless steel or other medical grade medals. In certain embodiments the levers 140, springs 150, gears 160 and gear tracks 170 can be prepared from medical grade plastics or metals.

Attached to the needle housing 110 can be levers 140a, 140b that are affixed to a tension systems, such as springs 150a, 150b. The tension systems can include springs, resistant plastics, coils, or other systems for providing tension. The tension is employed to rotate the intravenous needle after insertion into the patient. In one embodiment, the tension system is attached to the levers 140a, 140b. Additionally affixed to the levers 140a, 140b can be a gear track 170 and gears 160 that are in contact with the needle 120. The gears 160 are configured to move through gear track 170 and rotate the needle 120. The rotation of the needle 120 can be 180°. Alternative rotations can be from 90° to 270°. The optimal rotation angle is 180° which places the bevel of the needle 120 in the safest position to avoid accidental puncturing of the patient's vein. In an embodiment, the gears are configured to move within the gear track, and the gear track is configured to limit the rotation of the intravenous needle. In an embodiment, the bevel indicator is a first color when the rotation angle of the intravenous needle is 0° and a second color when the rotation angle of the intravenous needle is 180°.

The bevel of the needle 120 is designed for maximum efficiency for insertion into the vein. Generally, the bevel extends downward from the top of the needle edge to the bottom of the needle edge creating a downward bevel. Rotation of the needle 120 by 180° reverses the angle of the bevel and reduces the chance of accidental puncturing of the patient's vein.

In the embodiment shown in FIG. 1, the levers 140a, 140b are affixed to a back portion of the needle housing 110 on opposite sides of the needle housing 110. Alternative configurations can be employed with a single lever 140 or more than two levers 140a, 140b, 140c. The levers 140a, 140b are configured to be actuated by the fingers of the administrator, preferably the thumb and index finger, however, alternative styles of finger placement are envisioned by the present invention. The levers 140a, 140b can extend from a back portion of the needle housing 110 towards a front portion of the needle housing 110. Preferably the levers 140a, 140b will not extend beyond the front portion of the needle housing 110. Embodiments of the levers 140a, 140b can have ridges or grips to aid the hand and/or fingers of the administrator. To actuate the rotation of the needle 120, the levers 140a, 140b can be compressed towards the needle housing 110 which engages the gears 160 and gear track 170 and rotates the needle 120. The levers 140a, 140b, gears 160 and gear tracks 170 are configured to rotate the needle 120 180°.

The tension system, herein springs 150a, 150b are configured to provide tension to the levers 140a, 140b to hold them in an open or uncompressed position prior to engagement by the administrator to move the levers 140a, 140b into a closed or compressed position. When the levers 140a, 140b are in the closed position the needle 120 will have been rotated 180°.

As discussed above, alternative angles of rotation can be employed and the size and shape of the levers 140a, 140b, gears 160 and gear tracks 170 can be varied to provide alternative angles of rotation of the needle 120.

A bevel indicator can be included in the needle housing 110 that can show and/or indicate the angle of the bevel of the needle in the starting position) (0°) and finally in the rotated position) (180°).

FIG. 2 illustrates a side view of a rotating intravenous needle and catheter 200, according to an embodiment. As shown in FIG. 2 the lever 240a extends from a back portion of the needle housing 210 to a front portion of the needle housing 210. The length and width of the lever 140a can be varied in accordance with the size and shape of the needle housing and approximate size of the fingers of the administrator.

Additionally shown in FIG. 2 is a catheter release tab 235 configured to allow the administrator to release or disengage the catheter 230 from the needle housing 210 after the needle 220 has been inserted and rotated in the patient. The catheter release tab 235 is configured to be actuated by pressure from the thumb or index finger of the administrator thereby disengaging the catheter 230 from the needle housing 210. The catheter release tab can projects upwards from the catheter above the needle housing 210 to allow the administrator to easily disengage the catheter from the needle housing with one hand and without releasing the grip on the needle housing.

Embodiments can include a device including a rotating intravenous needle and catheter including a needle housing which can be formed from medical grade plastics in a cylindrical geometry. An intravenous needle prepared from stainless steel, wherein said intravenous needle can further includes a bevel at a forward edge of the intravenous needle. A catheter can be prepared from medical grade plastics, wherein the catheter can be configured to surround the intravenous needle. Levers can further be configured to actuate gears attached to the intravenous needle. The levers can be attached to the needle housing, wherein the levers can be affixed to a tension system configured to provide tension to the levers to resist compression of the levers. The levers can be configured to actuate gears affixed to the intravenous needle, and the gears can be configured to rotate the intravenous needle 180°.

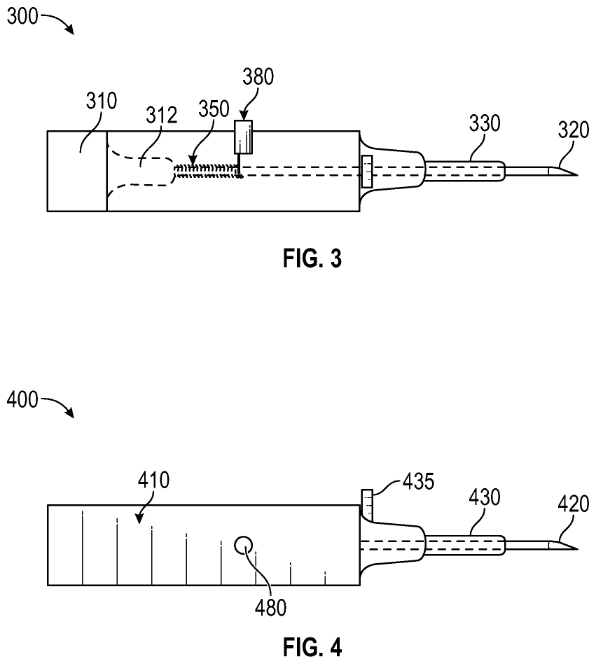

FIG. 3 illustrates a top view of an alternative rotating intravenous needle and catheter 300, according to an embodiment. As shown in FIG. 3 the rotating intravenous needle and catheter 300 can include an internal tension system 350 affixed to the needle 320. The internal tension system 350 can provide tension via springs, deformable plastics, coils, or other tension systems. In the embodiment shown in FIG. 3 the tension system 350 is a spring with preloaded tension. The tension system 350 is connected to an actuator button 380 that extends from the tension system 350 through the needle housing 310 and is accessible to the administrator. The actuator button 380 is configured to be depressed and release the tension in the tension system 350 and rotate the needle 320 by 180°. As discussed above alternative tension systems can be prepared that rotate the needle various rotations between 90° and 270°. The actuator button 380 can include ridges or grip to provide support to the fingers of the administrator.

The actuator button 380 is configured to be actuated by the thumb or index finger of the administrator. The size and location of the actuator button 380 can be varied in the needle housing 310 to accommodate different hand sizes of the administrator.

In the embodiment shown in FIG. 3 the tension system 350 extends from an internal needle anchor 312 positioned within the needle housing 310 towards a rear portion of the needle housing 310. The tension system 350 extends forward towards the front portion of the needle housing 310. In an embodiment the tension system 350 extends at least as far as the portion of the actuator button 380 that is connected to the tension system 350.

FIG. 4 illustrates a side view of an alternative rotating intravenous needle and catheter 400, according to an embodiment. Additionally illustrated in FIG. 4 is the catheter release tab 435 that is configured to allow the administrator to release the catheter 430 after the needle 420 has been inserted and rotated. Also shown in FIG. 4 is the top of the actuator button 480. The actuator button 480 when compressed will release the pre-loaded tension in the tension system (not shown) and allow the needle to rotate 180°.

An embodiment can include A system including a rotating intravenous needle and catheter including a needle housing and an intravenous needle, wherein said intravenous needle can further include a bevel at a forward edge of the intravenous needle. A catheter, wherein the catheter can be configured to surround the intravenous needle. An actuator button can be affixed to a tension system, and the tension system can be affixed to the intravenous needle. The actuator button and tension system can be configured to rotate the intravenous needle 180°.

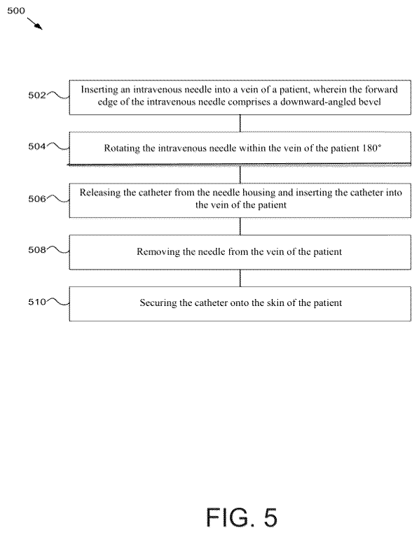

FIG. 5 illustrates a block chart of a method 500 of administering a rotating intravenous needle and catheter to a patient, according to an embodiment.

A method 500 of administering a rotating intravenous needle and catheter to a patient can include the following steps. Step 502 inserting an intravenous needle into a vein of a patient, wherein the forward edge of the intravenous needle comprises a downward angled bevel. Step 504 rotating the intravenous needle within the vein of the patient 180°, wherein the rotation is actuated via a tension system and a rotation actuator. Step 506 releasing the catheter from the needle housing and inserting the catheter into the vein of the patient. Step 508 removing the needle from the vein of the patient. Step 510 securing the catheter onto the skin of the patient.

A feature illustrated in one of the figures may be the same as or similar to a feature illustrated in another of the figures. Similarly, a feature described in connection with one of the figures may be the same as or similar to a feature described in connection with another of the figures. The same or similar features may be noted by the same or similar reference characters unless expressly described otherwise. Additionally, the description of a particular figure may refer to a feature not shown in the particular figure. The feature may be illustrated in and/or further described in connection with another figure.

Elements of processes (i.e. methods) described herein may be executed in one or more ways such as by a human, by a processing device, by mechanisms operating automatically or under human control, and so forth. Additionally, although various elements of a process may be depicted in the figures in a particular order, the elements of the process may be performed in one or more different orders without departing from the substance and spirit of the disclosure herein.

The foregoing description sets forth numerous specific details such as examples of specific systems, components, methods and so forth, in order to provide a good understanding of several implementations. It will be apparent to one skilled in the art, however, that at least some implementations may be practiced without these specific details. In other instances, well-known components or methods are not described in detail or are presented in simple block diagram format in order to avoid unnecessarily obscuring the present implementations. Thus, the specific details set forth above are merely exemplary. Particular implementations may vary from these exemplary details and still be contemplated to be within the scope of the present implementations.

Related elements in the examples and/or embodiments described herein may be identical, similar, or dissimilar in different examples. For the sake of brevity and clarity, related elements may not be redundantly explained. Instead, the use of a same, similar, and/or related element names and/or reference characters may cue the reader that an element with a given name and/or associated reference character may be similar to another related element with the same, similar, and/or related element name and/or reference character in an example explained elsewhere herein. Elements specific to a given example may be described regarding that particular example. A person having ordinary skill in the art will understand that a given element need not be the same and/or similar to the specific portrayal of a related element in any given figure or example in order to share features of the related element.

It is to be understood that the foregoing description is intended to be illustrative and not restrictive. Many other implementations will be apparent to those of skill in the art upon reading and understanding the above description. The scope of the present implementations should, therefore, be determined with reference to the appended claims, along with the full scope of equivalents to which such claims are entitled.

The foregoing disclosure encompasses multiple distinct examples with independent utility. While these examples have been disclosed in a particular form, the specific examples disclosed and illustrated above are not to be considered in a limiting sense as numerous variations are possible. The subject matter disclosed herein includes novel and non-obvious combinations and sub-combinations of the various elements, features, functions and/or properties disclosed above both explicitly and inherently. Where the disclosure or subsequently filed claims recite “a” element, “a first” element, or any such equivalent term, the disclosure or claims is to be understood to incorporate one or more such elements, neither requiring nor excluding two or more of such elements.

As used herein “same” means sharing all features and “similar” means sharing a substantial number of features or sharing materially important features even if a substantial number of features are not shared. As used herein “may” should be interpreted in a permissive sense and should not be interpreted in an indefinite sense. Additionally, use of “is” regarding examples, elements, and/or features should be interpreted to be definite only regarding a specific example and should not be interpreted as definite regarding every example. Furthermore, references to “the disclosure” and/or “this disclosure” refer to the entirety of the writings of this document and the entirety of the accompanying illustrations, which extends to all the writings of each subsection of this document, including the Title, Background, Brief description of the Drawings, Detailed Description, Claims, Abstract, and any other document and/or resource incorporated herein by reference.

As used herein regarding a list, “and” forms a group inclusive of all the listed elements. For example, an example described as including A, B, C, and D is an example that includes A, includes B, includes C, and also includes D. As used herein regarding a list, “or” forms a list of elements, any of which may be included. For example, an example described as including A, B, C, or D is an example that includes any of the elements A, B, C, and D. Unless otherwise stated, an example including a list of alternatively-inclusive elements does not preclude other examples that include various combinations of some or all of the alternatively-inclusive elements. An example described using a list of alternatively-inclusive elements includes at least one element of the listed elements. However, an example described using a list of alternatively-inclusive elements does not preclude another example that includes all of the listed elements. And, an example described using a list of alternatively-inclusive elements does not preclude another example that includes a combination of some of the listed elements. As used herein regarding a list, “and/or” forms a list of elements inclusive alone or in any combination. For example, an example described as including A, B, C, and/or D is an example that may include: A alone; A and B; A, B and C; A, B, C, and D; and so forth. The bounds of an “and/or” list are defined by the complete set of combinations and permutations for the list.

Where multiples of a particular element are shown in a FIG., and where it is clear that the element is duplicated throughout the FIG., only one label may be provided for the element, despite multiple instances of the element being present in the FIG. Accordingly, other instances in the FIG. of the element having identical or similar structure and/or function may not have been redundantly labeled. A person having ordinary skill in the art will recognize based on the disclosure herein redundant and/or duplicated elements of the same FIG. Despite this, redundant labeling may be included where helpful in clarifying the structure of the depicted examples.

The Applicant(s) reserves the right to submit claims directed to combinations and sub-combinations of the disclosed examples that are believed to be novel and non-obvious. Examples embodied in other combinations and sub-combinations of features, functions, elements and/or properties may be claimed through amendment of those claims or presentation of new claims in the present application or in a related application. Such amended or new claims, whether they are directed to the same example or a different example and whether they are different, broader. narrower or equal in scope to the original claims, are to be considered within the subject matter of the examples described herein.

1. A device, comprising:
a rotating intravenous needle and catheter comprising:
an needle housing formed from medical grade plastics in a cylindrical geometry,
an intravenous needle prepared from stainless steel,
wherein said intravenous needle further comprises a bevel at a forward edge of the intravenous needle,
a catheter prepared from medical grade plastics,
wherein the catheter is configured to surround the intravenous needle, and levers configured to actuate gears attached to the intravenous needle,
wherein:
the levers are attached to the needle housing,
the levers are affixed to a tension system configured to provide tension to the levers to resist compression of the levers,
said levers are configured to actuate gears affixed to the intravenous needle, and
the gears are configured to rotate the intravenous needle 180°.

2. The device of claim 1, wherein the tension system comprises springs configured to resist the compression of the levers and control the rotation of the intravenous needle.

3. The device of claim 1, further comprising a bevel indicator,
wherein the bevel indicator is configured to show an angle of rotation of the intravenous needle.

4. The device of claim 1, wherein the levers are attached to a rear portion of the needle housing and are configured to be compressed downward towards the needle housing via a thumb and index finger of an administrator.

5. The device of claim 1, wherein the levers further comprise ridges or grips configured to provide support for a hand and fingers of an administrator.

6. The device of claim 1, wherein the catheter further comprises:
a catheter release tab configured to allow an administrator to release the catheter from the needle housing via pressure from a thumb or index finger of the administrator.

7. The device of claim 1, wherein:
the gears are configured to move within a gear track, and
the gear track is configured to limit the rotation of the intravenous needle.

8. The device of claim 1, wherein:
a first lever is affixed to the needle housing, and
a second lever is affixed to an opposite side of the needle housing.

9. A method, comprising:
administering a rotating intravenous needle and catheter to a patient comprising:
inserting an intravenous needle into a vein of a patient,
wherein a forward edge of the intravenous needle comprises a downward angled bevel,
rotating the intravenous needle within the vein of the patient 180°,
wherein the rotation is actuated via a tension system and a rotation actuator,
releasing the catheter from a needle housing and inserting the catheter into the vein of the patient,
removing the needle from the vein of the patient; and
securing the catheter onto skin of the patient.

10. The method of claim 9, wherein:
the rotation actuator comprises:
a lever attached to a needle housing, wherein:
a tension system is attached between the lever and the needle housing, and
compression of the lever actuates gears configured to rotate the intravenous needle.

11. The method of claim 9, wherein:
the rotation actuator comprises:
an actuator button, wherein:
the tension system is attached to the intravenous needle,
the actuator button is attached to the tension system, and
compression of the actuator button releases preloaded tension in the tension system and rotates the intravenous needle.

12. The method of claim 10, wherein the lever is configured to be compressed by a thumb or index finger of an administrator.

13. The method of claim 11, wherein actuator button is configured to be compressed by a thumb or index finger of an administrator.

14. The method of claim 9, wherein:
a bevel indicator is located on the needle housing, and
the bevel indicator is configured to show the rotation angle of the bevel of the intravenous needle before and after rotation.

15. The method of claim 14, wherein the bevel indicator is a first color when the rotation angle of the intravenous needle is 0° and a second color when the rotation angle of the intravenous needle is 180°.

16. A system, comprising:
a rotating intravenous needle and catheter comprising:
a needle housing,
an intravenous needle,
wherein said intravenous needle further comprises a bevel at a forward edge of the intravenous needle,
a catheter,
wherein the catheter is configured to surround the intravenous needle,
an actuator button affixed to a tension system, and
a tension system affixed to the intravenous needle,
wherein the actuator button and tension system are configured to rotate the intravenous needle 180°.

17. The system of claim 16, further comprising:
a bevel indicator located on the needle housing,
wherein:
the bevel indicator is configured to show a rotation angle of the bevel of the intravenous needle before and after rotation, and
the bevel indicator is a first color when the rotation angle of the intravenous needle is 0° and a second color when the rotation angle of the intravenous needle is 180°.

18. The system of claim 16, wherein:
the actuator button is configured to be depressed by a thumb or index finger of an administrator, and
the actuator button further comprises ridges or grips to provide support for a thumb or index finger of the administrator.

19. The system of claim 16, wherein:
the tension system includes springs, and
the springs are preloaded with tension configured to rotate the intravenous needle 180°.

20. The system of claim 16, further comprising:
a catheter release tab, wherein:
the catheter release tab is configured to allow an administrator to release the catheter from the needle housing via pressure from a thumb or index finger of the administrator, and
the catheter release tab projects upwards from the catheter above the needle housing.
